(19) United States
(12) Patent Application Publication
(10) Pub. No.: US 2008/0194584 A1
(43) Pub. Date: Aug. 14, 2008

(54) Nitrogen Heteroaromatic Compounds Which Bind To The Active Site Of Protein Kinase Enzymes

(76) Inventors: Veronique Birault, Saffron Walden (GB); Jose Antonio Bravo, Suffolk (GB); Roger Crossley, Addisham (GB)

Correspondence Address: KLAUBER & JACKSON, 411 HACKENSACK AVENUE, HACKENSACK, NJ, 07601, US

(21) Appl. No.: 11/667,697

(22) PCT Filed: Nov. 11, 2005
(86) PCT No.: PCT/GB05/04358

§ 371 Date: Oct. 9, 2007

(30) Foreign Application Priority Data
Nov. 12, 2004 (GB) 0425026.2
Jan. 7, 2005 (GB) 0500245.6

(51) Int. Cl. A61K 31/444; C07D 401/04; A61K 31/4418; C07D 213/36; G01N 33/53; A61P 9/00; A61P 35/00; A61P 19/00; A61K 31/4709; C07D 401/14; A61K 31/4545; A61K 31/497

(52) U.S. Cl. 514/255.05; 546/257; 514/334; 514/357; 546/329; 435/4; 514/314; 546/152; 546/193; 514/318; 544/405

(57) ABSTRACT

Compounds of Formula (I)

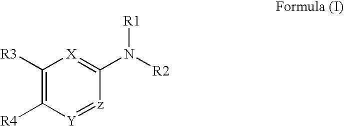

or pharmaceutically acceptable salts, hydrates, solvates, geometrical isomers, tautomers, optical isomers, or prodrug forms thereof, wherein X, Y, Z, R1, R2, R3 and R4 are as defined herein are capable of binding to the active site of protein kinase enzymes. In particular, they are inhibitors of a serine/threonine kinase more particularly Rho kinase (ROK, ROCK). The compounds can be used in methods of treatment and in the manufacture of medicaments for application to a number of therapeutic indications including cardiovascular disease (coronary vasospasm, hypertensive disease, arteriosclerosis), stroke, cancer, erectile dysfunction, asthma, osteoporosis, AIDS or an ocular condition including glaucoma, age related macular degeneration, lacrimal gland disease, or diabetic retinopathy, or suppression of neurite growth and hence a condition requiring nerve cell extension and connectivity, neuronal regeneration, inducing new axonal growth and promotion of axonal (re)wiring, repairing damage to neurons in the CNS caused by trauma (eg stroke, traumatic brain injury etc.) or neurodegeneration (eg Alzheimer's, Parkinson's etc), repair and recovery from and treatment of disorders such as spinal cord injury and in reducing the subsequent effects thereof, or pain caused by nerve cell damage such as following trauma or amputation for example in the treatment of neuropathic pain.

The present invention relates to a compound and a group of compounds capable of binding to the active site of protein kinase enzymes. In particular, the invention relates to a compound and a group of compounds which are inhibitors of a serine/threonine kinase more particularly Rho kinase (ROK, ROCK). In addition, the invention relates to methods of treatment and use of the compounds in the manufacture of a medicament for application to a number of therapeutic indications including cardiovascular disease (coronary vasospasm, hypertensive disease, arteriosclerosis), stroke, cancer, erectile dysfunction, asthma, osteoporosis, AIDS or an ocular condition including glaucoma, age related macular degeneration, lacrimal gland disease, or diabetic retinopathy, or suppression of neurite growth and hence a condition requiring nerve cell extension and connectivity, neuronal regeneration, inducing new axonal growth and promotion of axonal (re)wiring, repairing damage to neurons in the CNS caused by trauma (eg stroke, traumatic brain injury etc.) or neurodegeneration (eg Alzheimer's, Parkinson's etc), repair and recovery from and treatment of disorders such as spinal cord injury and in reducing the subsequent effects thereof, or pain caused by nerve cell damage such as following trauma or amputation for example in the treatment of neuropathic pain. The compounds can be used in screening programmes against protein kinases. The invention also provides methods for making compounds and libraries that include these compounds.

### BACKGROUND

The Kinase Gene Family

Protein kinases are a family of enzymes that catalyse the phosphorylation of hydroxyl groups in proteins. Approximately 2% of the genes encoded by the human genome are predicted to encode protein kinases. The reversible phosphorylation of specific tyrosine, serine, or threonine residues on a target protein can dramatically alter its function in several ways including activating or inhibiting enzymatic activity; creating or blocking binding sites for other proteins; altering subcellular localisation or controlling protein stability. Consequently protein kinases are pivotal in the regulation of a wide variety of cellular processes, including metabolism, cell proliferation, differentiation and survival. Of the many different cellular functions known to require the actions of protein kinases, some represent targets for therapeutic intervention for certain disease states.

One of the principal mechanisms by which cellular regulation is effected is through the transduction of extracellular signals across the membrane that, in turn, modulate biochemical pathways within the cell. Protein phosphorylation represents one course by which intracellular signals are propagated from molecule to molecule resulting finally in a cellular response. These signal transduction cascades are highly regulated and often overlapping as evidenced by the existence of many protein kinases as well as phosphatases. It is currently believed that a number of disease states and/or disorders are a result of either aberrant activation or functional mutations in the molecular components of kinase cascades. In humans, protein tyrosine kinases are known to have a significant role in the development of many disease states including diabetes, cancer and have also been linked to a wide variety of congenital syndromes. Serine threonine kinases also represent a class of enzymes, inhibitors of which are likely to have relevance to the treatment of cancer, diabetes and a variety of inflammatory cardiovascular disorders and AIDS.

Three potential mechanisms for inhibition of protein kinases have been identified thus far. These include a pseudo-substrate mechanism, an adenine mimetic mechanism and the locking of the enzyme into an inactive conformation by using surfaces other than the active site. The majority of inhibitors identified/designed to date act at the ATP-binding site. Such ATP-competitive inhibitors have demonstrated selectivity by virtue of their ability to target the more poorly conserved areas of the ATP-binding site.

Modulation of protein kinase activity therefore represents an attractive area for the design of new therapeutic agents. Protein kinases therefore represent a targeted intervention point in the treatment of a wide range of diseases.

Rho Kinases (ROK)

The Rho family of small GTP binding proteins contains at least 10 members including Rho A-E and G, Rac 1 and 2, Cdc42, and TC10. The effector domains of RhoA, RhoB, and RhoC have the same amino acid sequence appear to have similar intracellular targets. Rho kinase operates as a primary downstream mediator of Rho and exists as two isoforms α (ROCK2) and β (ROCK1).

ROK has a catalytic (kinase) domain in its N-terminal domain, a coiled-coil domain in its middle portion, and a putative pleckstrin-homology (PH) domain in its C-terminal domain. The Rho-binding domain of ROK is localized in the C-terminal portion of the coiled-coil domain and the binding the GTP-bound form of Rho results in enhancement of kinase activity. Numerous substrates of this kinase have been identified: myosin-binding subunit of myosin light-chain phosphatase; ERM (ezrin, radixin, moesin); adducin; intermediate filament (vimentin); the Na⁺—H⁺-exchanger, and LIM-kinase.

The Rho/Rho-kinase-mediated pathway plays an important role in the signal transduction initiated by many agonists, including angiotensin II, serotonin, thrombin, endothelin-1, norepinephrine, platelet-derived growth factor, ATP/ADP and extracellular nucleotides, and urotensin II. Through the modulation of its target effectors/substrates ROK plays an important role in various cellular functions including smooth muscle contraction, actin cytoskeleton organization, cell adhesion and motility and gene expression.

Therapeutic Potential of ROK Inhibitors

The apparent contribution of ROK to the pathogenesis of certain disorders has highlighted this kinase as a target for therapeutic intervention in a number of disease areas. The first generation ROK inhibitor, fasudil and the more recent Y-27632 compound has provided proof of concept in a variety of model systems.

Rho-kinase inhibitors have potential utility for the treatment of disorders caused by vascular smooth muscle hyper-constriction, including cerebral vasospasm, coronary vasospasm and hypertension. The beneficial effects of fasudil in the inhibition of cerebral and coronary vasospasm have been documented and there is accumulating evidence that ROK is involved in the pathogenesis of such events. ROK levels of expression and activity are significantly enhanced prior to development of symptoms in spontaneously hypertensive rats suggesting that this kinase is also involved in the pathogenesis of hypertension. Furthermore, short-term administration of Y-27632 preferentially reduces systemic blood pressure in various models of systemic hypertension.

By virtue of ROK's role in mediating a number of cellular functions perceived to be associated with the pathogenesis of arteriosclerosis, inhibitors of this kinase may also be useful for the treatment or prevention of various arteriosclerotic cardiovascular diseases, including angina pectoris, myocardial infarction, hypertensive vascular disease, stroke, heart failure, and arteriosclerosis obliterans. ROK has also been shown to be involved in endothelial contraction and enhancement of endothelial permeability which is thought to progress atherosclerosis.

The strategy of inhibiting ROK may also be useful for the treatment of other disorders associated with smooth muscle hyper-reactivity, such as bronchial asthma and glaucoma. Indeed, it has been recently demonstrated that ROK is involved in bronchial smooth muscle contraction and the regulation of aqueous humor outflow.

ROK is also thought to play a role in the negative regulation of bone marrow formation and that its inhibition may prove to be an appropriate new strategy for treatment of osteoporosis. Based upon rat model data, ROK inhibitors may also be useful for treatment of erectile dysfunction resulting from cavernosal smooth muscle relaxation. ROK inhibitors have also been implicated in treatment of AIDS through the proposed inhibition of HIV replication.

Inhibitors of this kinase have also been strongly implicated in the future treatment of cancer. It is known that constitutive activation of the Rho/ROK pathway contributes to the Ras transformation phenotype and mutations of Ras are thought to occur in as many as 25% of human tumors. Indeed pharmacological inhibition of ROK has been demonstrated to reduce both focus formation generated by Ras mutants and anchorage-independent growth in some colorectal cell lines. Evidence also exists to support a critical role for ROK in tumor cell invasion. To this end a ROK therapeutic has the potential for broad applicability to a wide range of cancer types.

Rho kinase is involved in the suppression of neurite growth and hence nerve cell extension and connectivity. Rho kinase inhibitors therefore have uses in neuronal regeneration, inducing new axonal growth and in promotion of axonal (re)wiring. They can therefore help in repairing damage to neurons in the CNS caused by trauma (eg stroke, traumatic brain injury etc.) or neurodegeneration (eg Alzheimer's, Parkinson's etc.). Rho kinase inhibitors are also useful in the repair and recovery from and treatment of disorders such as spinal cord injury and in reducing the subsequent effects thereof.

Such inhibitors are also of use in treatment of pain caused by nerve cell damage such as following trauma or amputation for example in the treatment of neuropathic pain.

In summary the early generation ROK inhibitors have shown promising efficacy in a variety of disease areas. The development of further ROK inhibitors with improved activity, selectivity and pharmacokinetic profiles is therefore needed to fully exploit the clinical potential of this target.

The invention addresses or ameliorates at least one of the disadvantages of the prior art, or provides a useful alternative.

Statement of Invention

In a first aspect the invention provides a compound of Formula (I)

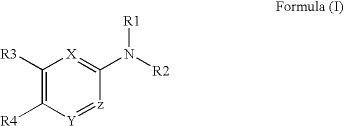

wherein:

X, Y, Z which may be the same or different are either
nitrogen; or
carbon substituted with hydrogen, hydroxy, halogen, trifluoromethyl, amino, C₁₋₆-aminoalkyl, C₁₋₆-alkyl, C₁₋₆-alkoxy, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl;

R1 and R2 which may be the same or different, and not to be both hydrogen, are
hydrogen,
aryl-C₁₋₆-alkyl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryloxy optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryl-C₁₋₆-alkyl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryloxy optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
CO—C₁₋₆-alkylaryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
CO-aryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
SO₂-aryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
C₁₋₆-alkyl optionally independently substituted with one or more of C₁₋₆-alkoxy, aryl, heteroaryl, C₃₋₈-cycloalkyl, C₃₋₈-cycloalkylamine, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
C₃₋₈-cycloalkyl optionally independently substituted with one or more of C₁₋₆-alkoxy, C₁₋₆-alkyl, C₁₋₆-alkylaryl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
alkenyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl;
alkynyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heterocyclyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl.

R3 and R4 which may be the same or different, and not to be both hydrogen, are
hydrogen,
heteroaryl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylhydroxy, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryl-C₁₋₆-alkyl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryloxy optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryl-C₁₋₆-alkyl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryloxy optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
C₁₋₆-alkyl optionally independently substituted with one or more of C₁₋₆-alkoxy, aryl, heteroaryl, C₃₋₈-cycloalkyl, C₃₋₈-cycloalkylamine, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
   C₃₋₈-cycloalkyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
alkenyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl;
alkynyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heterocyclyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl
   or a pharmaceutically acceptable salt, hydrate, solvate, geometrical isomer, tautomer, optical isomer, or prodrug form thereof.

In a second aspect the invention provides a method for making a compound according to a first aspect of the invention, which method comprises at least one step or a series of consecutive steps from the scheme defined herein below.

In a third aspect the invention provides a group of at least two compounds comprising or consisting of a set of structurally related compounds having the general formula I.

In a fourth aspect the invention provides a method for making a group of compounds according to an aspect of the invention, which method comprises at least one step or a series of consecutive steps from the scheme defined herein below.

In a further aspect the invention provides an assay comprising a group of compounds, or one or more compounds according to the invention.

In a further aspect the invention provides use of an assay according to an embodiment of the invention for identifying a compound that has therapeutic affect.

In a further aspect the invention provides a pharmaceutical composition that comprises a compound according to an embodiment of the invention or a compound identified in an assay according to an embodiment of the invention.

In a further aspect the invention provides a compound according to an embodiment of the invention for use in therapy.

In a further aspect the invention provides use of a compound according to an embodiment of the invention in the manufacture of a medicament for treatment or prophylaxis of a condition characterised by abnormal kinase activity.

In a further aspect the invention provides use of a compound according to an embodiment of the invention in the manufacture of a medicament for treatment or prophylaxis of a condition selected from cardiovascular disease (coronary vasospasm, hypertensive disease, arteriosclerosis), stroke, cancer, erectile dysfunction, asthma, osteoporosis, AIDS or an ocular condition including glaucoma, age related macular degeneration, lacrimal gland disease or diabetic retinopathy, or suppression of neurite growth and hence a condition requiring nerve cell extension and connectivity, neuronal regeneration, inducing new axonal growth and promotion of axonal (re)wiring, repairing damage to neurons in the CNS caused by trauma (eg stroke, traumatic brain injury etc.) or neurodegeneration (eg Alzheimer's, Parkinson's etc), repair and recovery from and treatment of disorders such as spinal cord injury and in reducing the subsequent effects thereof, or pain caused by nerve cell damage such as following trauma or amputation for example in the treatment of neuropathic pain.

In a further aspect the invention provides a method of treatment of a condition characterised by abnormal kinase activity that comprises administering a pharmaceutically effective amount of a compound according to an embodiment of the invention.

In a further aspect the invention provides a method of treatment of a condition selected from cardiovascular disease (coronary vasospasm, hypertensive disease, arteriosclerosis), stroke, cancer, erectile dysfunction, asthma, osteoporosis, AIDS or an ocular condition including glaucoma, age related macular degeneration, lacrimal gland disease or diabetic retinopathy, or suppression of neurite growth and hence a condition requiring nerve cell extension and connectivity, neuronal regeneration, inducing new axonal growth and promotion of axonal (re)wiring, repairing damage to neurons in the CNS caused by trauma (eg stroke, traumatic brain injury etc.) or neurodegeneration (eg Alzheimer's, Parkinson's etc), repair and recovery from and treatment of disorders such as spinal cord injury and in reducing the subsequent effects thereof, or pain caused by nerve cell damage such as following trauma or amputation for example in the treatment of neuropathic pain that comprises administering a pharmaceutically effective amount of a compound according to an embodiment of the invention.

### DETAILED DESCRIPTION

In a preferred embodiment of a compound according to the invention, R1 is hydrogen or C₁₋₆-alkyl optionally independently substituted with one or more of C₁₋₆-alkoxy, aryl, heteroaryl, C₃₋₈-cycloalkyl, C₃₋₈-cycloalkylamine, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl.

More preferably, R1 is hydrogen.

In a preferred embodiment, R2 is aryl-C₁₋₆-alkyl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl.

In an alternative preferred embodiment, R2 is heterocyclyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl

More preferably, R2 is 4-chlorobenzyl, 3-hydroxybenzyl or 4-chloro, 3-fluorobenzyl.

In a preferred embodiment, R3 is hydrogen, aryl or heteroaryl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylhydroxy, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl.

More preferably, when X, Y═N, Z=C, R3 is hydrogen.

In a preferred embodiment, R4 is hydrogen, aryl or heteroaryl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylhydroxy, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl.

More preferably, when X═C, Y═N, Z=C, R4 is hydrogen.

Most preferably a compound according to an embodiment of the invention has the structure of a compound of Table A below.

Any known compound having a structural formula identical to any one of the compounds covered by the formulae of scaffolds and permitted substitutions described herein is hereby explicitly disclaimed per se.

Preferably, an embodiment of a group of compounds according to the invention comprises compounds according to the first aspect of the invention, and said group of compounds has all or substantially all of the permitted substitutions represented by compounds therein.

Definitions

The following definitions shall apply throughout the specification and the appended claims.

Unless otherwise stated or indicated, the term “lower alkyl” denotes a straight or branched alkyl group having from 1 to 6 carbon atoms (“C₁₋₆-alkyl”). Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “Halo-C₁₋₆-alkyl” means a C₁₋₆-alkyl group substituted with one or more halogen atoms. Likewise, “aryl-C₁₋₆-alkyl” means a C₁₋₆-alkyl group substituted with one or more aryl groups.

Unless otherwise stated or indicated, the term “C₃₋₈-cycloalkyl” denotes a cyclic alkyl group having a ring size from 3 to 8 carbon atoms. Examples of said cycloalkyl include cyclopropyl, cyclobutyl, cyclopentyl, cyclohexyl, methylcyclohexyl, cycloheptyl, and cyclooctyl. For parts of the range “C₃₋₈-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₈-cycloalkyl, C₄₋₇-cycloalkyl, C₄₋₆-cycloalkyl, C₄₋₅-cycloalkyl, C₅₋₇-cycloalkyl, C₆₋₇-cycloalkyl, etc.

Unless otherwise stated or indicated, the term “C₁₋₆ alkoxy” denotes a straight or branched alkoxy group having from 1 to 6 carbon atoms. Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term “alkenyl” means a straight chain or branched alkenyl radical of 2 to 6 carbon atoms and containing one or more carbon-carbon double bonds and includes but is not limited to ethylene, n-propyl-1-ene, n-propyl-2-ene, isopropylene, etc.

Unless otherwise stated or indicated, the term “alkynyl” means a straight chain or branched alkynyl radical of 2 to 6 carbon atoms and containing one or more carbon-carbon triple bonds and includes but is not limited to ethynyl, 2-methylethynyl etc.

Unless otherwise stated or indicated, the term “aryl” refers to a 3-10 membered hydrocarbon ring system having at least one aromatic ring or being fused to one or more saturated or unsaturated rings including, but not limited to phenyl, pentalenyl, indenyl, indanyl, isoindolinyl, chromanyl, naphthyl, fluorenyl, anthryl, phenanthryl and pyrenyl. The aryl rings may optionally be substituted, for example with C₁₋₆-alkyl. Examples of substituted aryl groups are benzyl and 2-methylphenyl. Likewise, aryloxy refers to an aryl group bonded to an oxygen atom.

Unless otherwise stated or indicated, the term “heteroaryl” refers to a 3-10 membered hydrocarbon ring system having at least one aromatic ring which contains at least one heteroatom such as O, N, or S. Examples of heteroaryl groups include furyl, pyrrolyl, thienyl, oxazolyl, isoxazolyl, imidazolyl, thiazolyl, isothiazolyl, pyridinyl, pyrimidinyl, quinazolinyl, indolyl, pyrazolyl, pyridazinyl, quinolinyl, benzofuranyl, dihydrobenzofuranyl, benzodioxolyl, benzodioxinyl, benzothiazolyl, benzothiadiazolyl, and benzotriazolyl groups. The heteroaryl rings may optionally be substituted, for example with C₁₋₆-alkyl.

“Heterocyclyl” means a 3-10 membered ring system containing one or more heteroatoms selected from N, O or S and includes heteroaryl. The heterocyclyl system can contain one ring or may be fused to one or more saturated or unsaturated rings; the heterocyclyl can be fully saturated, partially saturated or unsaturated and includes but is not limited to heteroaryl and heterocarbocyclyl. Examples of carbocyclyl or heterocyclyl groups include but are not limited to cyclohexyl, phenyl, acridine, benzimidazole, benzofuran, benzothiophene, benzoxazole, benzothiazole, carbazole, cinnoline, dioxin, dioxane, dioxolane, dithiane, dithiazine, dithiazole, dithiolane, furan, imidazole, imidazoline, imidazolidine, indole, indoline, indolizine, indazole, isoindole, isoquinoline, isoxazole, isothiazole, morpholine, napthyridine, oxazole, oxadiazole, oxathiazole, oxathiazolidine, oxazine, oxadiazine, phenazine, phenothiazine, phenoxazine, phthalazine, piperazine, piperidine, pteridine, purine, pyran, pyrazine, pyrazole, pyrazoline, pyrazolidine, pyridazine, pyridine, pyrimidine, pyrrole, pyrrolidine, pyrroline, quinoline, quinoxaline, quinazoline, quinolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, and trithiane.

Unless otherwise stated or indicated, the term “halogen” shall mean fluorine, chlorine, bromine or iodine.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug form” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15.

The following abbreviations have been used:

HPLC means high performance liquid chromatography.

Clinical Use

The compounds of the formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulfuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, compounds of the invention can be incorporated into pharmaceutical compositions suitable for administration. Such compositions typically comprise at least one compound of the invention and at least one pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavoring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, ‘chlorobutanol, phenol, ascorbic acid, thimerosal, and the like.

In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum mono stearate and gelatin.

Sterile injectable solutions can be prepared by incorporating the active compound (e.g., a compound according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Experimental Methods

All reagents were commercial grade and were used as received without further purification, unless otherwise specified. Commercially available anhydrous solvents were used for reactions conducted under inert atmosphere. Reagent grade solvents were used in all other cases, unless otherwise specified.

Preparation of Compounds

General Method for Synthesising Compounds of Formula (I)

To a solution of the amine (0.3 mmol), acid (0.3 mmol) and diisopropylethylamine (0.45 mmol) in THF was added HBTU (0.33 mmol) and the reaction mixture stirred at room temperature overnight. It was then worked up and purified by column chromatography to give the desired intermediate.

To a solution of the desired intermediate in DMF (0.3 mmol, 0.5 ml) was added under nitrogen a solution of boronic acid in DMF (0.36 mmol, 0.6 ml) and 1.5M Na₂CO₃ (aq.) solution (0.75 mmol, 0.5 ml). Two solutions of palladium acetate (95 mg) and triphenylphosphine (335 mg) in 1,4-dioxane (15 ml) were freshly prepared and placed in a sonication bath for 2 min. The palladium catalyst (0.3 ml) was then added to reaction mixture under nitrogen. The reaction mixture was heated at 80° C. with agitation for 16 h. The reaction mixtures were filtered and purified by preparative reverse phase HPLC or purified on silicagel by flash chromatography.

The invention will now be described in detail with reference to specific examples of compounds and methods for their production.

Within this specification embodiments have been described in a way that enables a clear and concise specification to be written, but it will be appreciated that embodiments may be variously combined or separated without parting from the invention.

A compound according to an embodiment of the invention may be provided as a salt, preferably as a pharmaceutically acceptable salt of compounds of formula I. Examples of pharmaceutically acceptable salts of these compounds include those derived from organic acids such as acetic acid, malic acid, tartaric acid, citric acid, lactic acid, oxalic acid, succinic acid, fumaric acid, maleic acid, benzoic acid, salicylic acid, phenylacetic acid, mandelic acid, methanesulphonic acid, benzenesulphonic acid and p-toluenesulphonic acid, mineral acids such as hydrochloric and sulphuric acid and the like, giving methanesulphonate, benzenesulphonate, p-toluenesulphonate, hydrochloride and sulphate, and the like, respectively or those derived from bases such as organic and inorganic bases. Examples of suitable inorganic bases for the formation of salts of compounds for this invention include the hydroxides, carbonates, and bicarbonates of ammonia, lithium, sodium, calcium, potassium, aluminium, iron, magnesium, zinc and the like. Salts can also be formed with suitable organic bases. Such bases suitable for the formation of pharmaceutically acceptable base addition salts with compounds of the present invention include organic bases which are nontoxic and strong enough to form salts. Such organic bases are already well known in the art and may include amino acids such as arginine and lysine, mono-, di-, or trihydroxyalkylamines such as mono-, di-, and triethanolamine, choline, mono-, di-, and trialkylamines, such as methylamine, dimethylamine, and trimethylamine, guanidine; N-methylglucosamine; N-methylpiperazine; morpholine; ethylenediamine; N-benzylphenethylamine; tris(hydroxymethyl) aminomethane; and the like.

Salts of compounds according to an embodiment of the invention may be prepared in a conventional manner using methods well known in the art. Acid addition salts of said basic compounds may be prepared by dissolving the free base compounds according to the first or second aspects of the invention in aqueous or aqueous alcohol solution or other suitable solvents containing the required acid. Where a compound of the invention contains an acidic function, a base salt of said compound may be prepared by reacting said compound with a suitable base. The acid or base salt may separate directly or can be obtained by concentrating the solution e.g. by evaporation. The compounds of this invention may also exist in solvated or hydrated forms.

The invention also extends to prodrug of a compound according to an embodiment of the invention such as an ester or amide thereof. A prodrug is a compound that may be converted under physiological conditions or by solvolysis to a compound according to an embodiment of the invention or to a pharmaceutically acceptable salt of a compound according to an embodiment of the invention. A prodrug may be inactive when administered to a subject but is converted in vivo to an active compound of the invention.

A compound for use according to the invention may contain one or more asymmetric carbon atoms and may exist in racemic and optically active forms. All diastereomeric forms possible (pure enantiomers, tautomers, racemic mixtures and unequal mixtures of two or more enantiomers) are within the scope of the invention. Such compounds can also occur as cis- or trans-, E- or Z-double bond isomer forms. All isomeric forms and mixtures thereof are contemplated.

### EXAMPLES

Compounds of Formula I

Within the following table specific compounds according to the invention are exemplified and experimental data showing Rho kinase inhibitory activity is presented as +, ++, or +++ representing active, more active and very active based on assays conducted at 1-100 μM.

TABLE A

| ROK activity | |
|---|---|
| ++ | N-[3,4′]Bipyridinyl-5-yl-2-(4-chloro-phenyl)-acetamide |

| Example | Name | ROK activity |
|---|---|---|
| 1 | N-[3,4′]Bipyridinyl-5-yl-2-(4-chloro-phenyl)-acetamide | + |
| 2 | (4-Chloro-3-fluoro-benzyl)-(3-pyridin-4-yl-phenyl)-amine | +++ |
| 3 | (4-Chloro-3-fluoro-benzyl)-(3′-fluoro-[3,4′]bipyridinyl-5-yl)-amine | +++ |
| 4 | (2′-Chloro-[3,4′]bipyridinyl-5-yl)-(4-chloro-3-fluoro-benzyl)-amine | + |
| 5 | (3′-Chloro-[3,4′]bipyridinyl-5-yl)-(4-chloro-3-fluoro-benzyl)-amine | +++ |
| 6 | (4-Chloro-3-fluoro-benzyl)-(2′,3′-dichloro-[3,4′]bipyridinyl-5-yl)-amine | + |
| 7 | (4-Chloro-3-fluoro-benzyl)-(5-quinolin-5-yl-pyridin-3-yl)-amine | ++ |
| 8 | (4-Chloro-3-fluoro-benzyl)-(2′-chloro-3′-fluoro-[3,4′]bipyridinyl-5-yl)-amine | + |
| 9 | Benzyl-[3,4′]bipyridinyl-5-yl-amine | +++ |
| 10 | [3,4′]Bipyridinyl-5-yl-(2,6-dichloro-benzyl)-amine | + |
| 11 | [3,4′]Bipyridinyl-5-yl-(4-chloro-2-trifluoromethyl-benzyl)-amine | ++ |
| 12 | 1-[5-(4-Chloro-benzylamino)-[3,4′]bipyridinyl-2′-ylamino]-propan-2-ol | + |
| 13 | 3-([3,4′]Bipyridinyl-5-ylaminomethyl)-piperidine-1-carboxylic acid tert-butyl ester | + |
| 14 | 4-([3,4′]Bipyridinyl-5-ylaminomethyl)-piperidine-1-carboxylic acid tert-butyl ester | + |
| 15 | 4-([3,4′]Bipyridinyl-5-ylamino)-piperidine-1-carboxylic acid tert-butyl ester | + |
| 16 | [3,4′]Bipyridinyl-5-yl-piperidin-3-ylmethyl-amine | ++ |
| 17 | [3,4′]Bipyridinyl-5-yl-(3-trifluoromethyl-benzyl)-amine | ++ |
| 18 | [3,4′]Bipyridinyl-5-yl-(4-trifluoromethyl-benzyl)-amine | +++ |
| 19 | [3,4′]Bipyridinyl-5-yl-(4-chloro-2-fluoro-benzyl)-amine | +++ |
| 20 | N-[3,4′]Bipyridinyl-5-yl-4-chloro-benzamide | + |
| 21 | N-[3,4′]Bipyridinyl-5-yl-4-chloro-benzenesulfonamide | + |
| 22 | [3,4′]Bipyridinyl-5-yl-(2,4-difluoro-benzyl)-amine | +++ |
| 23 | (1-Benzyl-pyrrolidin-3-yl)-[3,4′]bipyridinyl-5-yl-amine | |
| 24 | (1-Benzyl-piperidin-4-yl)-[3,4′]bipyridinyl-5-yl-amine | |
| 25 | [3,4′]Bipyridinyl-5-yl-piperidin-4-ylmethyl-amine | |
| 26 | [3,4′]Bipyridinyl-5-yl-piperidin-4-yl-amine | |
| 27 | (1-Benzyl-piperidin-3-ylmethyl)-[3,4′]bipyridinyl-5-yl-amine | |
| 28 | [3,4′]Bipyridinyl-5-yl-pyrrolidin-3-yl-amine | |
| 29 | (2-Chloro-[3,4′]bipyridinyl-5-yl)-(4-chloro-3-fluoro-benzyl)-amine | |
| 30 | (4-Chloro-3-fluoro-benzyl)-(6-chloro-5-quinolin-5-yl-pyridin-3-yl)-amine | |
| 31 | 3-[(2-Chloro-[3,4′]bipyridinyl-5-ylamino)-methyl]-phenol | |
| 32 | 3-[(6-Chloro-5-quinolin-5-yl-pyridin-3-ylamino)-methyl]-phenol | |
| 33 | (4-Chloro-benzyl)-(2′-methoxy-[3,4′]bipyridinyl-5-yl)-amine | |
| 34 | [3,4′]Bipyridinyl-5-yl-(4-chloro-phenyl)-amine | |
| 35 | 5-([3,4′]Bipyridinyl-5-ylaminomethyl)-benzene-1,3-diol | |
| 36 | [3,4′]Bipyridinyl-5-yl-(3,5-difluoro-benzyl)-amine | |
| 37 | [3,4′]Bipyridinyl-5-yl-(2,3-difluoro-benzyl)-amine | |
| 38 | [3,4′]Bipyridinyl-5-yl-(3-fluoro-4-methyl-benzyl)-amine | |
| 39 | [3,4′]Bipyridinyl-5-yl-quinolin-6-ylmethyl-amine | |
| 40 | Benzofuran-5-ylmethyl-[3,4′]bipyridinyl-5-yl-amine | |
| 41 | [3,3′]Bipyridinyl-5-yl-(4-chloro-3-fluoro-benzyl)-amine | |
| 42 | N*5*-(4-Chloro-benzyl)-N*2′*-cyclopentyl-[3,4′]bipyridinyl-5,2′-diamine | |
| 43 | [3,4′]Bipyridinyl-5-yl-cyclopropylmethyl-amine | |
| 44 | [3,4′]Bipyridinyl-5-yl-quinolin-7-ylmethyl-amine | |
| 45 | (4-Chloro-3-fluoro-benzyl)-(6-pyridin-4-yl-pyrazin-2-yl)-amine | |
| 46 | 3-[(6-Pyridin-4-yl-pyrazin-2-ylamino)-methyl]-phenol | |
| 47 | 3-[(6-Quinolin-5-yl-pyrazin-2-ylamino)-methyl]-phenol | |

Examples of compounds of general formula (I) are recorded in Table B. Compounds were characterised by mass spectrometry using single quadrupole instrumentation with an electrospray source.

TABLE B

| Compound | Mol. Weight | MS data |
|---|---|---|
| N-[3,4′]Bipyridinyl-5-yl-2-(4-chloro-phenyl)-acetamide | 323.8 | M + 1 |

| | | | | Purity |
|---|---|---|---|---|
| Example | Name | Mol Weight | MS data | (%) |
| 1 | N-[3,4′]Bipyridinyl-5-yl-2-(4-chloro-phenyl)-acetamide | 323.7848 | M + 1 | 87.8 |
| 2 | (4-Chloro-3-fluoro-benzyl)-(3-pyridin-4-yl-phenyl)-amine | 312.77708 | M + 1 | 92.2 |
| 3 | (4-Chloro-3-fluoro-benzyl)-(3′-fluoro-[3,4′]bipyridinyl-5-yl)-amine | 331.75509 | M + 1 | 93.39 |
| 4 | (2′-Chloro-[3,4′]bipyridinyl-5-yl)-(4-chloro-3-fluoro-benzyl)-amine | 348.20969 | M + 1 | 98.68 |
| 5 | (3′-Chloro-[3,4′]bipyridinyl-5-yl)-(4-chloro-3-fluoro-benzyl)-amine | 348.20969 | M + 1 | 99.37 |
| 6 | (4-Chloro-3-fluoro-benzyl)-(2′,3′-dichloro-[3,4′]bipyridinyl-5-yl)- | 382.65472 | M + 1 | 96.24 |
| | amine | | | |
| 7 | (4-Chloro-3-fluoro-benzyl)-(5-quinolin-5-yl-pyridin-3-yl)-amine | 363.8252 | M + 1 | 100 |
| 8 | (4-Chloro-3-fluoro-benzyl)-(2′-chloro-3′-fluoro-[3,4′]bipyridinyl-5- | 366.20012 | M − 1 | 97.98 |
| | yl)-amine | | | |
| 9 | Benzyl-[3,4′]bipyridinyl-5-yl-amine | 261.3292 | M + 1 | 94.7 |
| 10 | [3,4′]Bipyridinyl-5-yl-(2,6-dichloro-benzyl)-amine | 330.21926 | M + 1 | 92.11 |
| 11 | [3,4′]Bipyridinyl-5-yl-(4-chloro-2-trifluoromethyl-benzyl)-amine | 363.77261 | M + 1 | 95.96 |
| 12 | 1-[5-(4-Chloro-benzylamino)-[3,4′]bipyridinyl-2′-ylamino]-propan- | 368.86957 | M + 1 | 97 |
| | 2-ol | | | |
| 13 | 3-([3,4′]Bipyridinyl-5-ylaminomethyl)-piperidine-1-carboxylic acid | 368.48291 | M + 1 | 98.81 |
| | tert-butyl ester | | | |
| 14 | 4-([3,4′]Bipyridinyl-5-ylaminomethyl)-piperidine-1-carboxylic acid | 368.48291 | M + 1 | 100 |
| | tert-butyl ester | | | |
| 15 | 4-([3,4′]Bipyridinyl-5-ylamino)-piperidine-1-carboxylic acid tert- | 354.45582 | M + 1 | 96.87 |
| | butyl ester | | | |
| 16 | [3,4′]Bipyridinyl-5-yl-piperidin-3-ylmethyl-amine | 268.3646 | M + 1 | 91.12 |
| 17 | [3,4′]Bipyridinyl-5-yl-(3-trifluoromethyl-benzyl)-amine | 329.32758 | M + 1 | 98.38 |
| 18 | [3,4′]Bipyridinyl-5-yl-(4-trifluoromethyl-benzyl)-amine | 329.32758 | M + 1 | 100 |
| 19 | [3,4′]Bipyridinyl-5-yl-(4-chloro-2-fluoro-benzyl)-amine | 313.76466 | M + 1 | 90 |
| 20 | N-[3,4′]Bipyridinyl-5-yl-4-chloro-benzamide | 309.75769 | M + 1 | 100 |
| 21 | N-[3,4′]Bipyridinyl-5-yl-4-chloro-benzenesulfonamide | 345.80994 | M + 1 | 100 |
| 22 | [3,4′]Bipyridinyl-5-yl-(2,4-difluoro-benzyl)-amine | 297.31006 | M + 1 | 96.67 |
| 23 | (1-Benzyl-pyrrolidin-3-yl)-[3,4′]bipyridinyl-5-yl-amine | 330.43629 | M + 1 | 97.76 |
| 24 | (1-Benzyl-piperidin-4-yl)-[3,4′]bipyridinyl-5-yl-amine | 344.46338 | M + 1 | 95.89 |
| 25 | [3,4′]Bipyridinyl-5-yl-piperidin-4-ylmethyl-amine | 268.3646 | M + 1 | 96.5 |
| 26 | [3,4′]Bipyridinyl-5-yl-piperidin-4-yl-amine | 254.33751 | M + 1 | 94.4 |
| 27 | (1-Benzyl-piperidin-3-ylmethyl)-[3,4′]bipyridinyl-5-yl-amine | 358.49047 | M + 1 | 97.44 |
| 28 | [3,4′]Bipyridinyl-5-yl-pyrrolidin-3-yl-amine | 240.31042 | M + 1 | 99.5 |
| 29 | (2-Chloro-[3,4′]bipyridinyl-5-yl)-(4-chloro-3-fluoro-benzyl)-amine | 348.20969 | M + 1 | 99.16 |
| 30 | (4-Chloro-3-fluoro-benzyl)-(6-chloro-5-quinolin-5-yl-pyridin-3-yl)- | 398.27023 | M + 1 | 92.3 |
| | amine | | | |
| 31 | 3-[(2-Chloro-[3,4′]bipyridinyl-5-ylamino)-methyl]-phenol | 311.77363 | M + 1 | 92.86 |
| 32 | 3-[(6-Chloro-5-quinolin-5-yl-pyridin-3-ylamino)-methyl]-phenol | 361.83417 | M + 1 | 97.23 |
| 33 | (4-Chloro-benzyl)-(2′-methoxy-[3,4′]bipyridinyl-5-yl)-amine | 325.80072 | M + 1 | 94.3 |
| 34 | [3,4′]Bipyridinyl-5-yl-(4-chloro-phenyl)-amine | 281.74714 | M + 1 | 97.51 |
| 35 | 5-([3,4′]Bipyridinyl-5-ylaminomethyl)-benzene-1,3-diol | 293.328 | M + 1 | 93.44 |
| 36 | [3,4′]Bipyridinyl-5-yl-(3,5-difluoro-benzyl)-amine | 297.31006 | M + 1 | 94.53 |
| 37 | [3,4′]Bipyridinyl-5-yl-(2,3-difluoro-benzyl)-amine | 297.31006 | M + 1 | 93.51 |
| 38 | [3,4′]Bipyridinyl-5-yl-(3-fluoro-4-methyl-benzyl)-amine | 293.34672 | M + 1 | 98.12 |
| 39 | [3,4′]Bipyridinyl-5-yl-quinolin-6-ylmethyl-amine | 312.37732 | M + 1 | 91.28 |
| 40 | Benzofuran-5-ylmethyl-[3,4′]bipyridinyl-5-yl-amine | 301.3509 | M + 1 | 99.02 |
| 41 | [3,3′]Bipyridinyl-5-yl-(4-chloro-3-fluoro-benzyl)-amine | 313.76466 | M + 1 | 96.4 |
| 42 | N*5*-(4-Chloro-benzyl)-N*2′*-cyclopentyl-[3,4′]bipyridinyl-5,2′- | 378.90841 | M + 1 | 94 |
| | diamine | | | |
| 43 | [3,4′]Bipyridinyl-5-yl-cyclopropylmethyl-amine | 225.29575 | M + 1 | 96.4 |
| 44 | [3,4′]Bipyridinyl-5-yl-quinolin-7-ylmethyl-amine | 312.37732 | M + 1 | 93.8 |
| 45 | (4-Chloro-3-fluoro-benzyl)-(6-pyridin-4-yl-pyrazin-2-yl)-amine | 314.75224 | M + 1 | 96.75 |
| 46 | 3-[(6-Pyridin-4-yl-pyrazin-2-ylamino)-methyl]-phenol | 278.31618 | M + 1 | 96.19 |
| 47 | 3-[(6-Quinolin-5-yl-pyrazin-2-ylamino)-methyl]-phenol | 328.37672 | M + 1 | 100 |

Preparation of Compounds

General Method A for Synthesising Compounds of Formula (I)

To a solution of the amine (0.3 mmol), acid (0.3 mmol) and diisopropylethylamine (0.45 mmol) in THF was added HBTU (0.33 mmol) and the reaction mixture stirred at room temperature overnight. It was then worked up and purified by column chromatography to give the desired intermediate.

To a solution of the desired intermediate in DMF (0.3 mmol, 0.5 ml) was added under nitrogen a solution of boronic acid in DMF (0.36 mmol, 0.6 ml) and 1.5M Na₂CO₃ (aq.) solution (0.75 mmol, 0.5 ml). Two solutions of palladium acetate (95 mg) and triphenylphosphine (335 mg) in 1,4-dioxane (15 ml) were freshly prepared and placed in a sonication bath for 2 min. The palladium catalyst (0.3 ml) was then added to reaction mixture under nitrogen. The reaction mixture was heated at 80° C. with agitation for 16 h. The reaction mixtures were filtered and purified by preparative reverse phase HPLC or purified on silicagel by flash chromatography.

Scheme for Synthesising Example 1 of Formula (I) Using General Method A

### Example 1

### N-[3,4′]Bipyridinyl-5-yl-2-(4-chloro-phenyl)-acetamide

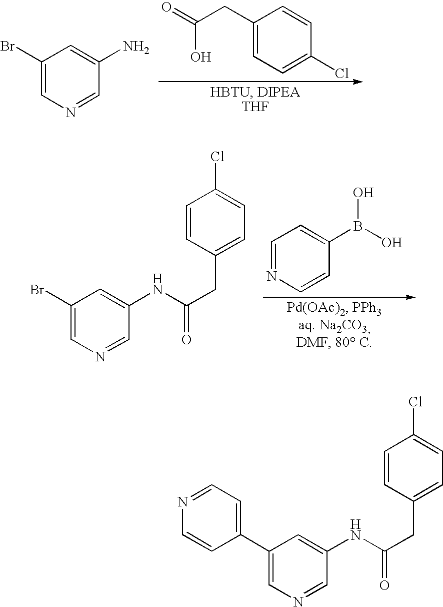

Scheme B for Synthesising Examples of Formula (I) Using General Method B

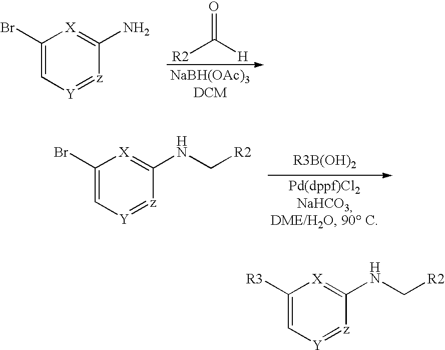

Reductive amination of aldehydes (R2) gives rise to the desired intermediates. These then undergo Suzuki reactions with boronic acids (R3) to give the final compounds of general formula (I).

General Method B

To the amine (1 eq) and aldehyde (1 eq) in dry DCM was added sodium triacetoxyborohydride (1.5 eq) and stirred at room temperature overnight. The reaction was then quenched with NaHCO₃ (aq), diluted with DCM, washed with water, dried (MgSO₄), evaporated and purified on silica gel by flash chromatography to give the desired intermediate.

To a solution of the intermediate (1 eq) in DME/H₂O (3:1) was added under a nitrogen atmosphere the boronic acid (2 eq) and NaHCO₃ (2 eq). Pd(dppf)Cl₂ (0.1 eq) was then added to the reaction mixture and heated at 90° C. stirring overnight. The reaction mixture was diluted with ethyl acetate, washed with water, dried (MgSO₄), evaporated and purified on silica gel by flash chromatography to give the desired product.

### Example 2

### (4-Chloro-3-fluoro-benzyl)-(3-pyridin-4-yl-phenyl)-amine

### Synthesis of the Intermediate: (3-Bromo-phenyl)-(4-chloro-3-fluoro-benzyl)-amine

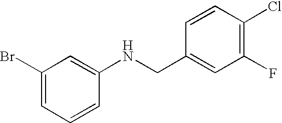

To 3-bromoaniline (1.58 g, 12.4 mmol) and 4-chloro-3-fluoro-benzaldehyde (1.93 g, 12.4 mmol) in dry DCM (75 ml) was added sodium triacetoxyborohydride (3.89 g, 18.6 mmol) and stirred at room temperature overnight. The reaction was then quenched with NaHCO₃ (aq), diluted with DCM, washed with water, dried (MgSO₄), evaporated and purified on silica gel by flash chromatography eluting with petroleum ether:ethyl acetate (1:1) to give (3-Bromo-phenyl)-(4-chloro-3-fluoro-benzyl)-amine (1.17 g). 1H (400 MHz, CDCl3) 4.18 (1H, bs, NH), 4.30 (2H, d, J=5.6 Hz), 6.49-6.50 (1H, m, Ar), 6.72-6.73 (1H, m, Ar), 6.85-6.98 (1H, m, Ar), 7.00-7.20 (3H, m, Ar), 7.30-7.50 (1H, m, Ar); m/z (ES): 312 (M−1).

### Synthesis of (4-Chloro-3-fluoro-benzyl)-(3-pyridin-4-yl-phenyl)-amine

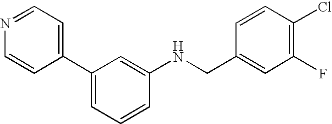

To a solution of (3-Bromo-phenyl)-(4-chloro-3-fluoro-benzyl)-amine (96.8 mg, 0.3 mmol) in DME/H₂O (3:1) (4 ml) was added under a nitrogen atmosphere the boronic acid (76 mg, 0.6 mmol) and NaHCO₃ (50 mg, 0.6 mmol). Pd(dppf)Cl₂ (24 mg, 0.03 mmol) was then added to the reaction mixture and heated at 90° C. stirring overnight. The reaction mixture was diluted with ethyl acetate, washed with water, dried (MgSO₄), evaporated then purified on silica gel by flash chromatography eluting with petroleum ether: ethyl acetate (1:1) to give the desired product. 1H (400 MHz, CDCl3) 4.30 (1H, bs, NH), 4.39-4.42 (2H, m), 6.60-6.70 (1H, m, Ar), 6.81-6.82 (1H, m, Ar), 6.98-7.00 (1H, m, Ar), 7.13-7.20 (2H, m, Ar), 7.35-7.52 (4H, m, Ar), 8.63 (2H, d J=5 Hz, Ar); m/z (ES) 279 (M+1).

### Example 3

### (4-Chloro-3-fluoro-benzyl)-(3′-fluoro-[3,4′]bipyridinyl-5-yl)-amine

### Synthesis of the Intermediate: (5-Bromo-pyridin-3-yl)-(4-chloro-3-fluoro-benzyl)-amine

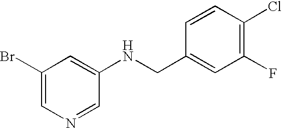

To 5-Bromo-pyridin-3-ylamine (2.2 g, 17.34 mmol) and 4-chloro-3-fluoro-benzaldehyde (2.75 g, 17.34 mmol) in dry DCM (25 ml) was added sodium triacetoxyborohydride (5.51 g, 26.01 mmol) and stirred at room temperature overnight. The reaction was then quenched with NaHCO₃ (aq), diluted with DCM, washed with water, dried (MgSO₄), evaporated and purified on silica gel by flash chromatography eluting with petroleum ether:ethyl acetate (1:1) to give (5-Bromo-pyridin-3-yl)-(4-chloro-3-fluoro-benzyl)-amine (2.16 g). 1H (400 MHz, CDCl3) 4.11-4.15 (1H, bs, NH), 4.31-4.33 (2H, m), 6.97-6.98 (1H, m, Ar), 7.06-7.15 (2H, m, Ar), 7.37-7.41 (1H, m, Ar), 7.95-7.96 (1H, m, Ar), 8.04 (1H, s, Ar); m/z 316 (M+1).

### Synthesis of (4-Chloro-3-fluoro-benzyl)-(3′-fluoro-[3,4′]bipyridinyl-5-yl)-amine

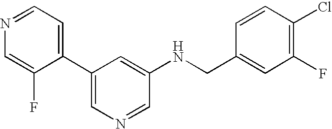

To a solution of (5-Bromo-pyridin-3-yl)-(4-chloro-3-fluoro-benzyl)-amine (100 mg, 0.32 mmol) in DME/H₂O (3:1) (8 ml) was added under a nitrogen atmosphere the boronic acid (83 mg, 0.47 mmol) and NaHCO₃ (53 mg, 0.64 mmol). Pd(dppf)Cl₂ (26 mg, 0.03 mmol) was then added to the reaction mixture and heated at 90° C. stirring overnight. The reaction mixture was diluted with ethyl acetate, washed with water, dried (MgSO₄), evaporated and purified on silica gel by flash chromatography eluting with DCM:MeOH (95:5) to give the desired product (31 mg). 1H (400 MHz, CDCl3) 4.35-4.42 (1H, bs, NH), 4.40-4.42 (2H, m), 7.05-7.20 (2H, m, Ar), 7.34-7.41 (2H, m, Ar), 8.11-8.12 (2H, m, Ar), 8.21 (1H, s, Ar), 8.47-8.49 (1H, m, Ar), 8.55 (1H, s, Ar); m/z: 332 (M+1).

| | Ingredients | mg/tablet |
|---|---|---|
| 1. | Active compound of formula (I) | 10.0 |
| 2. | Cellulose, microcrystalline | 57.0 |
| 3. | Calcium hydrogen phosphate | 15.0 |
| 4. | Sodium starch glycolate | 5.0 |
| 5. | Silicon dioxide, colloidal | 0.25 |
| 6. | Magnesium stearate | 0.75 |

The active ingredient 1 is mixed with ingredients 2, 3, 4 and 5 for about 10 minutes. The magnesium stearate is then added, and the resultant mixture is mixed for about 5 minutes and compressed into tablet form with or without film-coating.

It will be appreciated by those skilled in the art that the foregoing description is exemplary and explanatory in nature, and is intended to illustrate the invention and its preferred embodiments. Through routine experimentation, an artisan will recognize apparent modifications and variations that may be made without departing from the spirit of the invention. Thus, the invention is intended to be defined not by the above description, but by the following claims and their equivalents.

1. A compound of Formula (I) 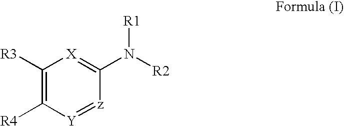
wherein:
X, Y, Z which may be the same or different are either
nitrogen; or
carbon substituted with hydrogen, hydroxy, halogen, trifluoromethyl, amino, C₁₋₆-aminoalkyl, C₁₋₆-alkyl, C₁₋₆-alkoxy, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl;
R1 and R2 which may be the same or different, and not to be both hydrogen are:
hydrogen; or
aryl-C₁₋₆-alkyl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryloxy optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryl-C₁₋₆-alkyl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryloxy optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
CO—C₁₋₆-alkylaryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
CO-aryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
SO₂-aryl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
C₁₋₆-alkyl optionally independently substituted with one or more of C₁₋₆-alkoxy, aryl, heteroaryl, C₃₋₈-cycloalkyl, C₃₋₈-cycloalkylamine, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
C₃₋₈-cycloalkyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
alkenyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl;
alkynyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heterocyclyl optionally independently substituted with one or more of C₁₋₆-alkoxy, C₁₋₆-alkyl, C₁₋₆-alkylaryl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl;
R3 and R4 which may be the same or different, and not to be both hydrogen, are
hydrogen; or
heteroaryl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylhydroxy, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryl-C₁₋₆-alkyl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heteroaryloxy optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryl-C₁₋₆-alkyl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
aryloxy optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
C₁₋₆-alkyl optionally independently substituted with one or more of C₁₋₆-alkoxy, aryl, heteroaryl, C₃₋₈-cycloalkyl, C₃₋₈-cycloalkylamine, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
C₃₋₈-cycloalkyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
alkenyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl;
alkynyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl; or
heterocyclyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl;
or a pharmaceutically acceptable salt, hydrate, solvate, geometrical isomer, tautomer, optical isomer, or prodrug form thereof.

2. A compound according to claim 1 wherein R1 is selected from hydrogen and C₁₋₆-alkyl; optionally wherein the C₁₋₆-alkyl is independently substituted with one or more of C₁₋₆-alkoxy, aryl, heteroaryl, C₃₋₈-cycloalkyl, C₃₋₈-cycloalkylamine, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl.

3. A compound according to claim 1 wherein R1 is hydrogen.

4. A compound according to claim 1 wherein R2 is (i) aryl-C₁₋₆-alkyl optionally independently substituted with one or more of methylenedioxy, hydroxy, C₁₋₆-alkylhydroxy, C₁₋₆-alkoxy, C₁₋₆-alkyl, trifluoromethyl, O—CF₃, halogen, CN, NO₂, aryl, heteroaryl, amino, C₁₋₆-aminoalkyl, CONR4R5, CO₂R4, CO₂H, NHCOR4, NR4R5, NHS(O)₂R4, NC(O)NR4R5, NC(O)OR4, OR4 or SR4; or (ii) heterocyclyl optionally independently substituted with one or more of C₁₋₆-alkoxy, C₁₋₆-alkyl, C₁₋₆-alkylamino, aryl, heteroaryl, CONR4R5, CO₂R4, CO₂H, NHCOR4, NR4R5, NHS(O)₂R4, NC(O)NR4R5, NC(O)OR4, OR4 or SR4; where R4 and R5 may be the same or different are either hydrogen, C₁₋₆-alkyl, C₁₋₆-alkoxy, C₁₋₆-alkylamino, aryl, hetero or heteroaryl.

5. A compound according to claim 1 wherein R2 is 4-chlorobenzyl, 3-hydroxybenzyl or 4-chloro, 3-fluorobenzyl.

6. A compound according to claim 1 wherein R3 is hydrogen or heteroaryl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylhydroxy, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 may be the same or different are either hydrogen, C₁₋₆-alkyl, C₁₋₆-alkoxy, C₁₋₆-alkylamino, aryl, hetero or heteroaryl.

7. A compound according to claim 1 wherein R3 is hydrogen, 4-pyridyl, 4-hydroxyphenyl, 3-benzamide or 5-quinoline.

8. A compound according to claim 1 wherein R4 is hydrogen or heteroaryl optionally independently substituted with one or more of methylenedioxy, hydroxy, C₁₋₆-alkylhydroxy, C₁₋₆-alkoxy, C₁₋₆-alkyl, amino, amino C₁₋₆-alkyl, amino C₁₋₆-alkylhydroxy, amino C₁₋₆-alkylaryl, amino C₃₋₈-cycloalkyl, halogen, aryl, heteroaryl, trifluoromethyl, O—CF₃, CN, NO₂, CONR4R5, CO₂R4, CO₂H, NHCOR4, NR4R5, NHS(O)₂R4, NC(O)NR4R5, NC(O)OR4, OR4 or SR4 where R4 and R5 maybe the same or different are either hydrogen, C₁₋₆-alkyl, C₁₋₆-alkoxy, C₁₋₆-alkylamino, aryl, hetero or heteroaryl.

9. A compound according to claim 1 wherein R4 is hydrogen.

10. A compound according to claim 1 wherein X, Y═N, Z=C, R3 is hydrogen or X═C, Y═N, Z=C, R4 is hydrogen.

11. A compound selected from the group consisting of:
N-[3,4′]Bipyridinyl-5-yl-2-(4-chloro-phenyl)-acetamide
(4-Chloro-3-fluoro-benzyl)-(3-pyridin-4-yl-phenyl)-amine
(4-Chloro-3-fluoro-benzyl)-(3′-fluoro-[3,4′]bipyridinyl-5-yl)-amine
(2′-Chloro-[3,4′]bipyridinyl-5-yl)-(4-chloro-3-fluoro-benzyl)-amine
(3′-Chloro-[3,4′]bipyridinyl-5-yl)-(4-chloro-3-fluoro-benzyl)-amine
(4-Chloro-3-fluoro-benzyl)-(2′,3′-dichloro-[3,4′]bipyridinyl-5-yl)-amine
(4-Chloro-3-fluoro-benzyl)-(5-quinolin-5-yl-pyridin-3-yl)-amine
(4-Chloro-3-fluoro-benzyl)-(2′-chloro-3′-fluoro-[3,4′]bipyridinyl-5-yl)-amine
Benzyl-[3,4′]bipyridinyl-5-yl-amine
[3,4′]Bipyridinyl-5-yl-(2,6-dichloro-benzyl)-amine
[3,4′]Bipyridinyl-5-yl-(4-chloro-2-trifluoromethyl-benzyl)-amine
1-[5-(4-Chloro-benzylamino)-[3,4′]bipyridinyl-2′-ylamino]-propan-2-ol
3-([3,4′]Bipyridinyl-5-ylaminomethyl)-piperidine-1-carboxylic acid tert-butyl ester
4-([3,4′]Bipyridinyl-5-ylaminomethyl)-piperidine-1-carboxylic acid tert-butyl ester
4-([3,4′]Bipyridinyl-5-ylamino)-piperidine-1-carboxylic acid tert-butyl ester
[3,4′]Bipyridinyl-5-yl-piperidin-3-ylmethyl-amine
[3,4′]Bipyridinyl-5-yl-(3-trifluoromethyl-benzyl)-amine
[3,4′]Bipyridinyl-5-yl-(4-trifluoromethyl-benzyl)-amine
[3,4′]Bipyridinyl-5-yl-(4-chloro-2-fluoro-benzyl)-amine
N-[3,4′]Bipyridinyl-5-yl-4-chloro-benzamide
N-[3,4′]Bipyridinyl-5-yl-4-chloro-benzenesulfonamide
[3,4′]Bipyridinyl-5-yl-(2,4-difluoro-benzyl)-amine
(1-Benzyl-pyrrolidin-3-yl)-[3,4′]bipyridinyl-5-yl-amine
(1-Benzyl-piperidin-4-yl)-[3,4′]bipyridinyl-5-yl-amine
[3,4′]Bipyridinyl-5-yl-piperidin-4-ylmethyl-amine
[3,4′]Bipyridinyl-5-yl-piperidin-4-yl-amine
(1-Benzyl-piperidin-3-ylmethyl)-[3,4′]bipyridinyl-5-yl-amine
[3,4′]Bipyridinyl-5-yl-pyrrolidin-3-yl-amine
(2-Chloro-[3,4′]bipyridinyl-5-yl)-(4-chloro-3-fluoro-benzyl)-amine
(4-Chloro-3-fluoro-benzyl)-(6-chloro-5-quinolin-5-yl-pyridin-3-yl)-amine
3-[(2-Chloro-[3,4′]bipyridinyl-5-ylamino)-methyl]-phenol
3-[(6-Chloro-5-quinolin-5-yl-pyridin-3-ylamino)-methyl]-phenol
(4-Chloro-benzyl)-(2′-methoxy-[3,4′]bipyridinyl-5-yl)-amine
[3,4′]Bipyridinyl-5-yl-(4-chloro-phenyl)-amine
5-([3,4′]Bipyridinyl-5-ylaminomethyl)-benzene-1,3-diol
[3,4′]Bipyridinyl-5-yl-(3,5-difluoro-benzyl)-amine
[3,4′]Bipyridinyl-5-yl-(2,3-difluoro-benzyl)-amine
[3,4′]Bipyridinyl-5-yl-(3-fluoro-4-methyl-benzyl)-amine
[3,4′]Bipyridinyl-5-yl-quinolin-6-ylmethyl-amine
Benzofuran-5-ylmethyl-[3,4′]bipyridinyl-5-yl-amine
[3,3′]Bipyridinyl-5-yl-(4-chloro-3-fluoro-benzyl)-amine
N*5*-(4-Chloro-benzyl)-N*2′*-cyclopentyl-[3,4′]bipyridinyl-5,2′-diamine
[3,4′]Bipyridinyl-5-yl-cyclopropylmethyl-amine
[3,4′]Bipyridinyl-5-yl-quinolin-7-ylmethyl-amine
(4-Chloro-3-fluoro-benzyl)-(6-pyridin-4-yl-pyrazin-2-yl)-amine
3-[(6-Pyridin-4-yl-pyrazin-2-ylamino)-methyl]-phenol, and
3-[(6-Quinolin-5-yl-pyrazin-2-ylamino)-methyl]-phenol, and mixtures thereof.

12. A method for making a compound according to claim 1 which comprises the steps of adding HBTU to a solution of amine, acid and diisopropylethylamine in THF, stirring at room temperature overnight then working up and purifying the desired intermediate. Followed by adding, under nitrogen, a solution of boronic acid in DMF and Na₂CO₃ (aq.) solution to a solution of the desired intermediate in DMF; placing palladium acetate and triphenylphosphine in 1,4-dioxane in a sonication bath; adding the palladium catalyst to the reaction mixture under nitrogen; heating with agitation; and purifying the compound.

13. A method for making a compound according to claim 1 which comprises the steps of adding sodium triacetoxyborohydride to a solution of the amine and aldehyde in dry DCM, stirring at room temperature overnight then working up and purifying the desired intermediate. Followed by adding the boronic acid and NaHCO₃, to a solution of the intermediate in DME/H₂O (3:1) under a nitrogen atmosphere followed by Pd(dppf)Cl₂, and heating the reaction mixture overnight then working up and purifying the product.

14. A group of at least two compounds comprising or consisting of a set of structurally related compounds of claim 1.

15. (canceled)

16. An assay comprising a group of compounds, or one or more compounds according to claim 1.

17. An assay according to claim 16 for identifying a compound that has therapeutic affect.

18. A pharmaceutical composition that comprises at least one compound according to claim 1, a group of compounds.

19. (canceled)

20. (canceled)

21. (canceled)

22. A method of treatment of a condition characterised by abnormal kinase activity that comprises administering a pharmaceutically effective amount of at least one compound according to claim 1.

23. A method of treatment according to claim 22 wherein the condition is selected from cardiovascular disease (coronary vasospasm, hypertensive disease, arteriosclerosis), stroke, cancer, erectile dysfunction, asthma, osteoporosis, AIDS or an ocular condition including glaucoma, age related macular degeneration, lacrimal gland disease or diabetic retinopathy, or a condition requiring suppression of neurite growth and hence a condition requiring nerve cell extension and connectivity, neuronal regeneration, inducing new axonal growth and promotion of axonal (re)wiring, repairing damage to neurons in the CNS caused by trauma (eg stroke, traumatic brain injury etc.) or neurodegeneration (eg Alzheimer's, Parkinson's etc), repair and recovery from and treatment of disorders such as spinal cord injury and in reducing the subsequent effects thereof, or pain caused by nerve cell damage such as following trauma or amputation for example in the treatment of neuropathic pain.

24. (canceled)
